(19) United States
(12) Patent Application Publication
(10) Pub. No.: US 2007/0203550 A1
(43) Pub. Date: Aug. 30, 2007

(54) Method and apparatus for application of light to tissue

(76) Inventor: Thomas Perez, Chicago, IL (US)

Correspondence Address: Orum & Roth LLC, Ste 1616, 53 W Jackson Blvd, Chicago, IL, 60604, US

(21) Appl. No.: 11/363,554

(22) Filed: Feb. 27, 2006

(51) Int. Cl. A61N 5/00

(52) U.S. Cl. 607/86; 600/102

(57) ABSTRACT

a light device that is used to irradiates mucous membrane, blood organ, tumor or other body tissue. The light device has a power supply the least one light source and a camera the light source admits light at least one therapeutic wavelength preferably UVA and or UVC light the device also has a light source capable of emitting infrared radiation. A treatment provider uses the camera to locate and identify tissue to the irradiated. The treatment provider then positions the tip of the device and irradiates the tissue.

### BACKGROUND OF THE INVENTION

1. Field of the Invention

The present invention relates to a method and apparatus for providing light to blood. More specifically, the present invention relates to a method and apparatus for irradiation to mucous and/or tumor cites.

2. Background of the Prior Art

Ultraviolet (UV) light can be used to treat a multitude of medical problems, including for example bacterial, viral and fungal infections, poisoning, fatigue, Alzheimer's disease, allergies and asthma, rheumatic diseases and arthritis, diabetes, hepatitis, and cancer. UV light sterilizes the blood and acts as an antibiotic.

In UV light therapy, a treatment provider generally illuminates the patient's skin or blood. If the UV light is applied to the skin it is typically provided to the patient's skin either with a wrap or lamp. UV light is commonly used to treat jaundiced babies. Because infant's skin is thin and the blood vessels are close to the surface, UV light is typical applied to the skin when treating jaundiced babies.

Applying the UV light directly to a patient's blood supply is known as photoluminescence or UV blood illumination (UBI). UV blood illumination increases oxygen, destroys toxins and boosts the immune system. In prior art UBI, a small amount of blood is drawn from the patient, up to about 250 cc. The body has about 5.6 L of blood. The blood that is drawn travels through a cuvette or glass chamber. The blood is repeatedly illuminated with UV light and then returned to the body. The process is repeated, typically a day or several days later. These treatments are time consuming, and require regular trips to a medical facility. In addition, trained personal must be available to provide the treatments.

There is a need for alternative treatment methods. There is a need for an apparatus and method for treating other body organs, tissue or tumors with ultraviolet light therapy.

The capillaries in mucous membranes are close to the surface. These capillaries are very sensitive. Capillary exposure of the mucus membrane is significantly greater than other exposed body surfaces. The greater capillary exposure allows for greater penetration of the ultraviolet spectrum.

### SUMMARY OF THE INVENTION

The present invention is a light device that is used to irradiate mucous membrane, blood, organ, tumor or other body tissue. The light device has a battery or other power supply, at least one ultra-violet light source, and a camera. The light source emits light at one or more therapeutic wavelengths. Preferably, the light is UV light at one or more therapeutic wavelengths. Most preferably the UV light sources emit UV-A or UV-C light or a combination of UV-A and UV-C light. The device also optionally has a light source capable of emitting infrared radiation.

Fiber optic strand(s) run through a flexible tube to illuminate the mucous membrane, organ, tumor or other selected tissue. Capillaries are plentiful and close to the surface in the mucous membranes. Thus, illuminating the mucous membrane allows for blood illumination.

The camera is located at the flexible tube end with the one or more light sources and connected to a monitor.

The light device is used topically or inserted into a patient cavity, such as through the mouth or anus. A doctor or other treatment provider uses the camera to locate the light at a preferred location such as a tumor. The light device preferably has a light source that emits infrared light. The light source may be the same light source that emits the UV light or it may be a separate light source. The tumor or other selected site is illuminated with the IR light. Th and e IR light prepares the selected site. After being irradiated with IR radiation, the selected site is better able to absorb the therapeutic wavelengths of light. The IR light is turned off and the site is then illuminated with one or more therapeutic wavelengths of light. If more than one therapeutic wavelengths are being administered, they can be administered substantially contemporaneously or sequentially or a combination of contemporaneously and sequentially. The IR light is preferably applied before any of the UV radiation is applied. Optionally, IR light can be applied contemporaneously with the UV light or the UV and IR radiation can be alternately pulsed.

U.S. application Ser. No. 11/248995 filed 12 Oct. 2005; U.S. application Ser. No. 11/235652 filed 26 Sep. 2005; U.S. application Ser. No. 11/140272 filed 27 May 2005; U.S. application Ser. No. 11/140270 filed 27 May 2005; Ser. No. 11/140562 filed 27 May 2005; U.S. application Ser. No. 11/125,789 filed 10 May 2005; U.S. application Ser. No. 11/088613 filed 23 Mar. 2005; U.S. application Ser. No. 11/076169 filed 9 Mar. 2005; U.S. application Ser. No. 10/926209 filed 25 Aug. 2004; and U.S. Provisional Application No. 60/503,678 filed Sep. 17, 2003 are all hereby incorporated in their entirety.

### BRIEF DESCRIPTION OF THE DRAWINGS

FIG. 1 is a multi-frequency light guide assembly with camera.

FIG. 2 is a side view of a multi-frequency light guide assembly;

FIG. 3 is a cross section of a light device;

FIG. 4 is a portable light pack.

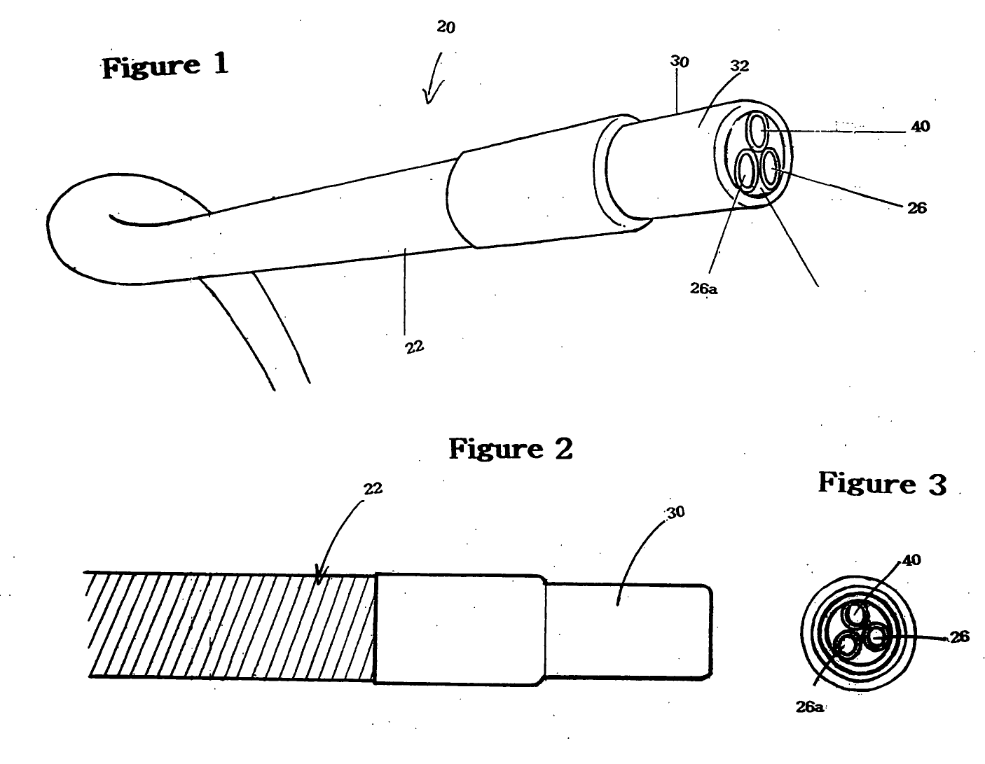

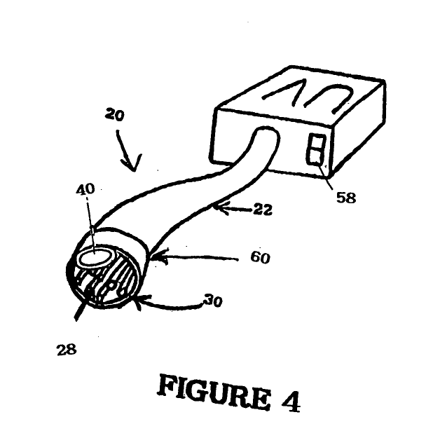

### DETAILED DESCRIPTION OF THE INVENTION

Light at one or more therapeutic wavelengths such as ultraviolet light (UV), is used to treat many diseases including infections, poisoning, fatigue, allergies, hepatitis, cancer and HIV. UV light increases the oxygen combining power of the blood, destroys toxins, viruses, fungi, bacteria, and boosts the immune system. UV light also sterilizes and acts as an antibiotic.

Preferably, UV light at one or more therapeutic wavelength is utilized in the present invention. More preferably either UV-A or UV-C light or a combination of UV-A and UV-C light is utilized in the present invention. For some conditions and/or diseases UV-A light is more effective than UV-C and for other conditions and/or diseases UV-C light is more effective than UV-A light. The wavelengths or wavelengths of light to be used to treat the patient are selected based on the wavelength or wavelength that will best treat the condition or disease of the patient. Optionally, the device also utilizes infrared light. IR irradiation prepares the tissue to absorb more of the therapeutic UV light.

The invention is a light device 20 that is used to illuminate a portion of the patient with a therapeutic wavelength(s), preferably UV light. The UV light may illuminate the patient's skin, mucous membrane, blood, organ, tumor or other tissue. The device comprises a power supply (not shown) to supply power to at least one light source. Preferably, the light device has two or more light sources. Light source 26 comprises a light guide tip that emits light, the light may be any therapeutic wavelength(s) such as UV-A and/or UV-C, the emitted light may also be IR, visible light, or any combination of desired wavelengths.

The device preferably further comprises a camera 40. Camera 40 is connected to a monitor (not shown). Camera 40 may be connected to monitor in a wired or wireless manner. Camera 40 is used by the treatment provider to locate and/or identify the tissue to be treated and to position the light source 26 prior to and/or during treatment.

Device 20 comprises a housing for the light source and the camera, preferably the housing 22 is a flexible tube having a tip 30. Preferably, tip is made of a transparent material or other material that allows emission of UV light. Optionally, tip 30 has a window the permits the emission of the UV radiation. Preferably, tip 30 is made of a non-rigid, semi-flexible or flexible material as this will be more comfortable for the patient. The tip 30 may be made of metal. There may be an epoxy sealer 32 or other sealant to keep bodily fluids from getting into tip 30.

Device 20 has a battery or other power supply or is connectable to a power supply (not shown).

Light source 26 is a liquid core light guide or other known light guide. In one embodiment, device light guide is made of rubber or other flexible tubing 26 and houses fiber optic strands 28. Alternatively, light source is LEDs. Light source may be fused silica fiber optic bundles housed in flexible tubing 22. Fused silica fiber optic bundles do not emit significant heat. Optionally light source 26 is a cold cathode fluorescent bulb. One or more low voltage cables may be used to power the light source.

Light source 26 emits UV light at a one or more therapeutic wavelength(s). More preferably, the light is UV-C light, UV-A light or a combination thereof. Alternatively, a plurality of LEDs, having one or more different wavelengths of light at one or more therapeutic wavelengths is used. It is preferable that a substantial portion of the emitted light be UV-C and/or UV-A. Preferably device 20 has light source 26a adapted to emit infrared radiation. Infrared radiation prepares the tissue to absorb the therapeutic wavelengths of light.

Emitted radiation travels to the end tip 30 of the flexible tubing and is emitted. Emitted radiation directly illuminates patient's tissue. Tubing may have a transparent cover 60 or window at the tip 30 in the patient.

Flexible tubing 22 is preferably made of rubber or other non-rigid medical grade material. Preferably, the material is flexible so that tubing can be inserted into an orifice of the patient, such as the nose, ear, mouth, vagina or rectum and then with the aid of a camera threaded until the tip is directed at the selected tissue.

Alternatively, the flexible tube 22 and tip 30 are inserted and the tissue treated laproscopically. In another alternative, patient has a port and is surgically implanted and tubing and tip are inserted through the port. In yet another alternative, device or a portion of device 20 such as a portion of flexible tubing 22 can be surgically implanted in patient.

Device 20 allows therapeutic wavelength(s) of light to be supplied directly to blood, organ, mucous membrane, tumor, or other tissue. Thus, infected or damaged tissue or tumor can be directly treated. For example, the digestive tract is coated with mucous membrane and us when the flexible tube is inserted through the mouth or anus, sensitive capillaries can be a radiated. Capillary exposure of the mucous membrane is significantly greater than other externally exposed body surfaces. Greater capillary exposure allows for greater penetration of the therapeutic ultraviolet spectrum.

Treatment provider can insert device into patient when a treatment is needed. Treatment provider views images provided by the camera on a monitor. Treatment provider uses the images to identify tissue to be sampled, biopsied or to receive treatment. The images allow the treatment provider to identify the tissue and to position the light guide. Preferably the selected tissue is first a radiated with infrared light. Then the selected tissue is a radiated with one or more therapeutic wavelengths such as UVA, UVC or a combination of UVA and UVC light.

Optionally, the device has a mechanism for collecting a tissue sample for example the device may have a mechanism for snipping off a piece of tissue or taking a core sample.

Device 20 could be turned on and off manually. Alternatively, device 20 could automatically turn off the infrared light and turn on the ultraviolet light at selected treatment times or durations. Further, the light source could automatically be turned off after a set treatment duration, such as twenty minutes.

Device 20 could have a controller such as a computer or other smart interface that limits the number of treatments given time period, limits the total amount of treatment time in a given time period, automatically provides treatments, pulses the light source, alternates between selected therapeutic wavelengths or between ultraviolet and infrared wavelengths or provides only particular wavelengths. The computer or other smart interface could keep a treatment record. The computer or other smart interface could communicate wirelessly, via the Internet or through other electronic means to automatically update the doctor's treatment records. Computer preferably can automatically adjust treatment time, wavelength or other factors based on patient input, doctor orders or other data.

Light device preferably has a manual on/off switch 58. Electronics preferably include a controller, a timer or smart interface such as a computer.

The computer or other smart interface could keep a treatment record. The computer or other smart interface could communicate wirelessly, via the Internet or through other electronic means to automatically update the doctor's treatment records. The computer could automatically adjust the treatment time based on input from the patient, the doctor, treatment records, or other data.

I claim:

1. A illumination device comprising:
a flexible tubing having a tip portion adapted to emit radiation;
a power supply;
at least one light source powered by said power supply and located in said housing; said at least one light source adapted to emit radiation at one or more therapeutic wavelengths through the tip portion.

2. The tissue illuminator of claim 1 wherein the light source is a cold cathode fluorescent bulb, a light emitting diode a fiber-optic bundle, a fused silica fiber optic bundle, or a cold cathode fluorescent bulb.

3. The tissue illuminator of claim 1 wherein the light source emits UV radiation.

4. The tissue illuminator of claim 3 wherein the UV radiation is UV-A radiation, UV-C radiation or a combination thereof.

5. The tissue illuminator of claim 2 wherein the light source is a medical grade UV light bulb.

6. The tissue illuminator of claim 1 further comprising a camera in said housing, said camera connected to a monitor.

7. The tissue illuminator of claim 3 further comprising a light source capable of emitting IR irradiation.

8. The tissue illuminator of claim 4 further comprising a light source capable of emitting IR irradiation.

9. The tissue illuminator of claim 3 further comprising a camera in said housing, said camera connected to a monitor.

10. The tissue illuminator of claim 7 further comprising a camera in said housing, said camera connected to a monitor.

11. The tissue illuminator of claim 1 wherein the tip is transparent and a second end portion of the flexible tubing is connected to the power supply

12. The tissue illuminator of claim 11 wherein a low voltage cable connects the power supply and the light source.

13. The tissue illuminator of claim 1 wherein the tip is made of a flexible material.

14. The tissue illuminator of claim 1 further comprising a controller.

15. The tissue illuminator of claim 1 further comprising an on/off switch.

16. The tissue illuminator of claim 14 wherein the controller automatically controls the at least light source.

17. The tissue illuminator of claim 14 wherein the controller automatically controls the light source by pulsing the therapeutic wavelengths, by automatically shutting off the IR light source, by automatically activating the IR light source; by automatically shutting off the UV light source, by automatically activating the UV light source; by automatically shutting off the UVA irradiation, by automatically activating the UVA irradiation; by automatically shutting off the UVC irradiation, by automatically activating the UVC irradiation; or by combinations thereof.

18. The tissue illuminator of claim 14 wherein the controller is adapted to select the wavelength to be emitted by the light source.

19. The-tissue illuminator of claim 14 wherein the controller is a computer.

20. The tissue illuminator of claim 19 wherein the computer is adapted to maintain and/or transmit treatment records.

21. A tissue illumination system comprising:
a tissue illuminator and a patient, said tissue illuminator comprising:
a flexible tubing having a tip portion adapted to emit radiation;
a power supply;
at least one light source powered by said power supply and located in said housing; said at least one light source adapted to emit radiation at one or more therapeutic wavelengths through the tip portion; and
a camera connected to a monitor.

22. A blood illumination system of claim 21 wherein the therapeutic wavelengths is one or more wavelengths of UV light.

23. A blood illumination system of claim 22 wherein the UV light is UV-A radiation, UV-C radiation or combinations thereof.

24. The tissue illuminator of claim 22 further comprising a light source capable of emitting IR irradiation.

25. The tissue illuminator of claim 23 further comprising a light source capable of emitting IR irradiation.

26. The blood illuminator of claim 21 wherein the camera is wirelessly connected to the monitor.

27. A method of illuminating tissue comprising the steps of:
inserting a portion of an illumination device in the patient;
viewing images of the patient on a monitor to identify the tissue to be illuminated;
identifying the tissue to be illuminated;
irradiating the issue with a therapeutic wavelength;
removing the portion of the illumination device.

28. The method of claim 27 further comprising the step of irradiating the tissue with a IR light source before irradiating it with one or more therapeutic wavelengths.

29. The method of claim 28 wherein the therapeutic wavelength is UVA, UVC or a combination of UVA and UVC.

30. The method of claim 28 further comprising the step of obtaining a tissue sample of the tissue.
